(19) United States
(12) Patent Application Publication
(10) Pub. No.: US 2008/0167272 A1
(43) Pub. Date: Jul. 10, 2008

(54) Thalidomide metabolites and methods of use

(75) Inventors: Lai-Ming Ching, Auckland (NZ); Brian D. Palmer, Auckland (NZ)

Correspondence Address: EDWARDS ANGELL PALMER & DODGE LLP, P.O. BOX 55874, BOSTON, MA, 02205, US

(73) Assignee: Auckland Uniservices Limited, Auckland (NZ)

(21) Appl. No.: 11/454,420

(22) Filed: Jun. 16, 2006

Related U.S. Application Data
(60) Provisional application No. 60/691,385, filed on Jun. 16, 2005.

(51) Int. Cl. A61K 31/661; A61K 31/4523; A61P 35/00

(52) U.S. Cl. 514/110; 514/339

(57) ABSTRACT

The present invention concerns certain hydrolytic thalidomide metabolites, combinations thereof with other anti-neoplastic compounds and their use in the treatment of solid tumours.

### CROSS-REFERENCE TO RELATED APPLICATIONS

This applications claims benefit of U.S. provisional application 60/691,385 filed Jun. 16, 2005, the entire contents of which is herein incorporated by reference.

### TECHNICAL FIELD

The present invention concerns certain hydrolytic thalidomide metabolites, combinations thereof with other anti-neoplastic compounds and their use in the treatment of solid tumours.

### BACKGROUND

Thalidomide (α-phthalimidoglutarimide) has a number of biological activities that, despite its teratogenicity (Mc Bride, 1961; Lenz, 1962) and removal from widespread clinical use as a sedative in the 1950s, has led to its re-emergence for the treatment of a number of inflammatory conditions, and more recently for the treatment of cancer.

Thalidomide's anti-tumour activities have been mainly attributed to its anti-angiogenic properties (D'Amato et al., 1994; Kenyon et al., 1997; Kruse et al., 1998; Joussen et al., 1999), although there is increasing evidence of the involvement of cytokine modulation (Kenyon et al., 1997; Hideshima et al., 2001a; Hideshima et al., 2001b; Dmoszynska et al., 2002; Tosi et al., 2002; Li et al., 2003). Metabolites have been suggested to be responsible for the anti-angiogenic effects of thalidomide (Gordon et al., 1981; Ng et al., 2003), and hydroxylated metabolites have been found to be effective in inhibiting angiogenesis in a number of different assays (Marks et al., 2002; Price et al., 2002; Ng et al., 2003). On the other hand, modulation of cytokine biosynthesis by thalidomide occurs efficiently without metabolic enzymes (Moreira et al, 1993) indicating that either thalidomide itself or a hydrolysis product is responsible for that activity.

Despite the recent successful applications of thalidomide in anti-tumour therapy, its use is still associated with significant side-effects. There is therefore a need for novel therapeutic agents and compositions with more desirable and advantageous properties.

The object of the present invention is to ameliorate one or more disadvantages of current anti-tumour therapies or to provide a useful alternative.

### SUMMARY OF THE INVENTION

According to a first aspect there is provided a composition comprising one or more thalidomide metabolites, wherein the one or more metabolites exhibit anti-tumour activity and optionally a pharmaceutically acceptable carrier.

According to a second aspect there is provided a composition comprising one or more thalidomide metabolites, wherein the one or more metabolites exhibit anti-angiogenic or cytokine modulatory activity and optionally a pharmaceutically acceptable carrier.

According to a third aspect there is provided a composition comprising one or more thalidomide metabolites, wherein the one or more metabolites exhibit anti-tumour, anti-angiogenic, or cytokine inhibitory activity, and at least one other anti-neoplastic compound, optionally in conjunction with a pharmaceutically acceptable carrier.

Preferably the thalidomide metabolites are hydrolysis products and more preferably the hydrolysis product is CG (N-(o-carboxybenzoyl)glutamic acid imide). The anti-tumour activity of a thalidomide metabolite is preferably activity against any solid vascularised tumours such as for example breast, lung, brain, colon, ovarian, prostate, testicular, uterine, liver and the like.

Preferably the anti-neoplastic compound is selected from the group consisting of DMXAA, cyclophosphamide or variants and analogues thereof. It will be understood however that other commonly used anti-neoplastic agents, such as the taxanes, platinum compounds, tubulin binders, top( )-isomerase poisons, anti-metabolites and vascular targeting agents can also be advantageously used in this combination.

According to a fourth aspect there is provided a method of treating a solid tumour comprising the administration to a subject requiring such treatment a composition of any one of the previous aspects.

Preferably the solid tumour to be treated can be colon cancers, breast cancers, liver cancers, lung cancers, brain tumours, ovarian tumours, prostate cancers, testicular cancers, uterine tumours and the like.

### BRIEF DESCRIPTION OF THE FIGURES

FIG. 1. Tumour growth delay in mice untreated, or treated with DMXAA or DMXAA combined with Thalidomide (Thal) or hydrolysis products/metabolites of Thal.

### Control

[symbol] DMXAA 25 mg/kg [symbol]
DMXAA 25 mg/kg+PG 100 mg/kg [symbol]
DMXAA 25 mg/kg+PiG 100 mg/kg [symbol]
DMXAA 25 mg/kg+CG 100 mg/kg [symbol]
DMXAA 25 mg/kg+Thal 100 mg/kg [symbol]
DMXAA 25 mg/kg+CG 20 mg/kg [symbol]
DMXAA 25 mg/kg+Thal 20 mg/kg [symbol]
DMXAA 25 mg/kg+CG 4 mg/kg [symbol]
DMXAA 25 mg/kg+Thal 4 mg/kg

FIG. 2. Colon 38 tumour volumes 21 days after treatment in mice. “*” represents significant difference (p<0.05, student's t-test) compared with DMXAA alone treatment.

FIG. 3. TNF levels in (A) serum and (B) tumour tissue of mice untreated, or treated with DMXAA alone or DMXAA combined with Thal or hydrolysis products/metabolites of Thal. “*” represents significant difference (p<0.05, student's t-test) compared with DMXAA alone treatment.

FIG. 4. TNF production by HPBL from seven healthy volunteers at different concentrations of LPS.

FIG. 5. The effect of Thal, CG, PG or PiG on LPS-induced TNF production by HPBL from healthy human volunteers. (A) TNF activity, (B) percentage of inhibition.

FIG. 6. Effects of Thal, CG, PG and PiG on tube formation of ECV 304 cells in Matrigel. Cells were treated with medium only, medium with vehicle only and indicated concentrations of drugs.

FIG. 7. Inhibition of the tube formation of ECV 304 cells in Matrigel by Thal and CG at different concentrations.

FIG. 8. CG and Thal concentrations in PBS solutions at different pH during 24 h of incubation at 37° C.

FIG. 9. Growth of Colon 38 tumours without treatment (filled circles), or following treatment with CG (100 mg/kg, open triangles), cyclophosphamide (220 mg/kg, open circles), or the combination of CG plus cyclophosphamide (filled triangles).

### DESCRIPTION OF THE PREFERRED EMBODIMENT

Our earlier studies on multiple myeloma patients treated with thalidomide could not detect the presence of hydroxylated thalidomide metabolites but only three hydrolysis metabolites, PG, PiG and CG. (see below for definition).

### PG:

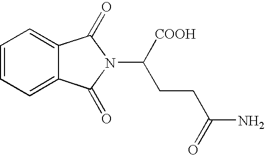 [0026] 5-Amino-2-(1,3-dioxo-1,3-dihydro-2H-isoindol-2-yl)-5-oxopentanoic acid or Phthaloylglutamine

### PiG:

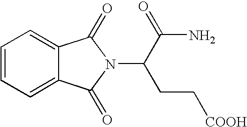 [0028] 5-Amino-4-(1,3-dioxo-1,3-dihydro-2H-isoindol-2-yl)-5-oxopentanoic acid or Phthaloylisoglutamine

### CG:

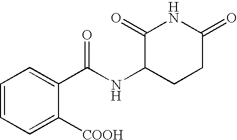 [0030] 2-{[(2,6-Dioxo-3-piperidinyl)amino]carbonyl}benzoic acid or
N-(o-Carboxybenzoyl)glutamic acid imide

For the thalidomide metabolites defined above, which contain a chiral center, the structures and definitions provided are intended to define the R-enantiomer and the S-enantiomer or a mixture of these two enantiomers.

The present invention is based in part on further studies which demonstrate the ability of certain thalidomide hydrolysis metabolites, in particular metabolite CG, to inhibit angiogenesis and to modulate cytokine production. It is further based on observations that certain hydrolysis metabolites can enhance the anti-tumour action of the anti-cancer drugs such as DMXAA and cyclophosphamide in an animal model. DMXAA and its analogues, variants and derivatives are described in U.S. Pat. No. 5,281,620, incorporated in its entirety herein by reference. Cyclophosphamide is a well known anti-neoplastic agent. Thus, synergistic anti-tumour combinations comprising CG and other anti-neoplastic compounds are an integral part of the invention described herein.

In one part the present invention employs an accepted animal model to demonstrate the use and efficacy of the compositions, including synergistic combinations, and methods in the treatment of a typical vascularised solid tumour. In the context of the present invention, the term “vascularised solid tumour” is used to describe any solid tumour, whether benign or cancerous, which relies on ample blood supply for its establishment and growth. Thus, the tissue or organ location, or tissue origin, of such tumours is not material to the compositions and methods of the present invention as long as there is reliance by the tumour on its blood supply. For example the compositions and methods of the present invention may be used to treat colon cancers, breast cancers, liver cancers, lung cancers, brain tumours, ovarian tumours, prostate cancers, testicular cancers, uterine tumours and the like.

In another part the present invention employs well recognized and accepted cell culture systems to demonstrate the anti-angiogenic and cytokine modulatory activities of the thalidomide metabolites and in particular metabolite CG.

The inventive concept will now be described in more detail with reference to non-limiting examples.

### EXAMPLES

### Experimental

### 1. Tumour Growth Delay Determinations

Mice (C57B1/6 bred at the Animal Resources Unit, University of Auckland, New Zealand) were implanted with Colon 38 tumours, obtained originally from the Mason Research Institute (Auckland, New Zewaland) by transferring 1 mm³ pieces of the tumour into a subcutaneous opening in the left flank of anaesthetized mice. The mice with tumours were used for growth delay experiments when tumours had reached approximately 3-5 mm in diameter. Mice were divided into 10 groups, an untreated control group, a group treated intraperitoneally (i.p.) with DMXAA (5,6-dimethylxanthenone-4-acetic acid, synthesized at the Auckland Cancer Society Research Centre—ACSRC (Auckland, New Zealand) at 25 mg/kg only, and 8 groups treated with DMXAA together with the hydrolytic thalidomide metabolites PG, PiG, CG also synthesized at the ACSRC as previously described (Lu et al, 2003), or thalidomide at 100 mg/kg, or together with CG or thalidomide at 20 mg/kg or 4 mg/kg. DMXAA was dissolved in saline at 2.5 mg/ml and injected i.p. into mice in a volume of 10 μl/g body weight. Thalidomide, PG, PiG and CG were dissolved in dimethylsulphoxide at 40, 8 or 1.6 mg/ml (for dosages of 100, 20, 4 mg/kg respectively) and injected i.p. in a volume of 2.5 μl/g body weight. Tumours were measured 2-3 times weekly after treatment and tumour volumes were calculated as 0.52×a²×b, where a and b are the minor and major axes of the tumour. The arithmetic means and standard errors were calculated for each point, counting cured animals as having zero tumour volume, and expressed as fractions of the pre-treatment volume. Growth delay was determined as the difference in the number of days required for the tumour to reach four times the pre-treatment volume. Mice in which the tumour had completely disappeared were kept for 3 months to ensure that the tumours did not re-grow.

### 2. Modulation of TNF Production in Mice

Mice with Colon 38 tumours, approximately 5-7 mm in diameter, were divided into groups of 5. One group was untreated. Other groups were treated with DMXAA (25 mg/kg) alone; DMXAA (25 mg/kg)+thalidomide (100 mg/kg), DMXAA (25 mg/kg)+PG (100 mg/kg), DMXAA (25 mg/kg)+PiG (100 mg/kg), DMXAA (25 mg/kg)+CG (100 mg/kg), DMXAA (25 mg/kg)+thalidomide (20 mg/kg), DMXAA (25 mg/kg)+CG (20 mg/kg), and thalidomide, PG, PiG or CG only at 100 and 200 mg/kg. Three hours after treatment, mice were anaesthetized with halothane (NZ Pharmacology Ltd., Christchurch, New Zealand), and blood was collected from the ocular sinus and tumours were excised. Blood was coagulated overnight at 4° C., and then centrifuged for 30 min at 2,000×g and 4° C., and the serum was removed and stored at −80° C. until use. Tumour tissues were weighed and homogenized in 1.5 ml α-minimal essential medium (Gibco, Grand Island, N.Y., USA). The homogenates were centrifuged (2,000×g, 30 min, 4° C.) and supernatant was removed and re-centrifuged (14,000×g, 30 min, 4° C.). The final supernatants were removed and kept at −80° C. until use. TNF levels in serum and supernatants derived from tumour homogenates were assayed for TNF as described in section 4 (below).

### 3. Modulation of TNF Production In Vitro

Blood (20 ml) each from seven healthy volunteers was collected into heparinized tubes, and was overlayed on top of 10 ml of Ficoll-Paque Plus (Amersham Biosciences AB, Uppsala, Sweden) in 50 ml Falcon polypropylene conical tubes (Becton Dickson Labware, Franklin Lakes, N.J.). The tubes were centrifuged for 30 min at 300×g and 4° C., and the layer of cells was removed and re-suspended at 1×10⁶ cells/ml in A-MEM medium supplemented with foetal bovine serum (Moregate BioTech Ltd, Hamilton, New Zealand) (10% v/v), streptomycin sulphate (100 μg/ml) and penicillin-G (100 units/ml) (Sigma, St Louis, Mo., USA). Cells were added to a 24-well plate (Nunc, Kamstrup, Roskilde, Denmark) with LPS (Escherichia coli serotype 055:B5; Sigma, St. Louis, Mo., USA) plus thalidomide or CG at 0.04, 0.2, 1, 5, 25 & 100 μg/ml; or PG or PiG at 1, 5 & 25 μg/ml in a final volume of 1 ml. Wells containing cells only and cells plus LPS only were included as controls. After 12 hours of incubation, supernatants were removed and assayed immediately for TNF as described in section 4.

### 4. TNF Assay

TNF was assayed using a commercially available ELISA kit (OpEIA human or murine TNF kit, PharMingen, San Diego, Calif., USA), according to the manufacturer's instructions. Briefly, samples (100 μl/well) in duplicate together with a serial dilution of TNF (31.25-2000 pg/ml) for the standard curve, were added to flat-bottom 96-well plates pre-coated with immobilised monoclonal anti-TNF antibody and incubated at room temperature for 2 h. The wells were then washed, biotinylated polyclonal antibody to TNF was added, followed by peroxidase-labelled streptavidin, and incubated at room temperature for 1 h. The wells were washed again, and the substrate, tetramethylbenzidine and hydrogen peroxide was added and after 10 min the reaction was stopped with 2 N sulphuric acid. The absorbance at 450 nm was determined using a microtitre plate reader.

### 5. Inhibition of Tube Formation In Vitro

Each well in a 24-well plate was coated with 300 μl of Matrigel (Becton Dickinson, San Jose, Calif., USA) which was allowed to solidified at 37° C. for 1 h. ECV304 cells (4×10⁴) (from American Type Culture Collection, CRL-1998, also known as T24) in a final volume of 1 ml of M-199 medium supplemented with 10% FBS, containing thalidomide, CG, PG and PiG at varying concentrations (2-25 μg/ml), were plated onto the Matrigel. After 15-18 h of incubation at 37° C. at 5% CO₂, the culture supernatant was aspirated. The cells were fixed and stained using Diff-Quick staining kit (Dade AG, Dudingen, Switzerland) according to the manufacturer's instructions. After removal of the staining solution the stained cells in each well were photographed along with a measuring ruler placed at the bottom edge of the photographing field (Olympus Camedia C-5050 professional digital camera, Olympus Optical, Tokyo, Japan). The total length of the tubes formed in the photographs was measured and divided by the area of the field (mm/mm²).

### 6. Cytotoxicity Assay

The 3-(4,5-dimethylthiazol-2-yl)-2,5-diphenyl tetrazolium bromide (MTT) assay, which measures mitochondrial metabolic activity, was used to determine cell viability (Mosmann, 1983). Cells (4×10³/well) were cultured with various concentrations of thalidomide, CG, PG and PiG in a final volume of 100 μl M199 medium (Gibco, Grand Island, N.Y., USA) supplemented with 10% foetal bovine serum (1,100 μg/ml streptomycin sulphate and 100 units/ml penicillin-G (Sigma, St Louis, Calif., USA) in flat-bottomed 96-well plates at 37° C. at 5% CO₂ for 18 h. At the end of the incubation period, 10 μl of MTT (Sigma, St. Louis, Mo., 5 mg/ml in PBS) was added to each well, and the cells were further incubated for 2 h. The supernatants were then removed and the formazan crystals in the cells were dissolved by the addition of 100 μl of DMSO per well, and the absorbance at 590 nm was read using a plate reader. Each concentration of drug was tested in triplicate wells, and the mean ±standard error of the mean (SEM) was calculated.

### 7. Stability Studies of CG In Vitro

CG (50 μM) with 60 μM of phenacetin (as an internal standard) at pH 3.5, 6.8, 7.1, 7.4, 7.7 and 8 in 8 mm clear polyethylene starburst conical snap vials (Alltech, Deerfield, Ill., USA), was incubated in the autosampler (Waters Associates, Milford, Mass., USA) at 37° C. Aliquots (50 μl) of the solutions were then automatically loaded at 0, 6, 12, 18 and 24 hours after incubation onto a Waters Breeze chromatograph (Waters Associates, Milford, Mass., USA) which consisted of a Model 717plus autosampler, Model 1525 binary pump and Model 2487 dual wavelength absorbance detector.

Compounds were separated using a previously described (Chung et al, 2004) HPLC assay for thalidomide using a 100×4.6 mm stainless steel Luna 5 μm Phenylhexyl column (Phenomenex, Torrance, Calif., USA) and a mobile phase (Milli Q water:acetonitrile 10:1.3 v/v) that was pumped at a flow rate of 2 ml/min. Thalidomide and phenacetin were detected at ultraviolet wavelengths of 220 and 248 nm, respectively. The retention times of phenacetin and thalidomide and were 13 and 11 min, respectively. Data acquisition and integration was achieved using Breeze™ Software, Milford, Calif., USA. Each HPLC run included standard curves of thalidomide (0.6-100 μM) or CG (0.2-50 μM) with phenacetin (60 μM) as the internal standard in PBS. Thalidomide in plasma at 0.6, 5, and 25 μM and CG in plasma at 0.2, 5, and 25 μM were stored at −80° C. and were included where appropriate as quality controls, and were found to be stable over a period of 14 days within 10% and 5% of the validated value respectively. The r² of calibration curves of thalidomide was 0.9995, and for CG calibration curves was 0.9999. The intra-assay recovery was 90-110% (n=8) and the CV was 5-9% (n=4), while the inter-assay recovery was 96-104% (n=15) and the CV was 2-4% (n=6).

To construct the calibration curve, the peak-area ratios to the internal standard were plotted against thalidomide concentration and the best-fit straight line was obtained by linear regression analysis. The correlation coefficient of the calibration curves had r² values of greater than 0.9995. The thalidomide/phenacetin peak ratio was determined for all samples and the concentration of thalidomide in each sample was determined from the calibration curve.

The intraassay accuracy and precision were acceptable with relative recoveries and coefficient of variation (CV) of 90-110% and 5-9% (n=8), respectively. Similar results were achieved for interassay accuracy and precision with relative recoveries and CV's of 96-104% and 2-4% (n=15). Quality control plasma and tissue homogenates with added three nominal thalidomide concentrations (0.6, 40, and 80 μM) were stored at −80° C. These were included in each analysis and were found to be stable over a period of 14 days and within 10% of the validated value (n=15).

The ratio of the area of the thalidomide peak or the CG peak to that of the phenacetin peak in the HPLC chromatographs then calculated and the concentration of thalidomide and CG was determined using the calibration curve. The percentage degradation of CG was calculated as: [1-(CG concentration after incubation/starting concentration)]×100.

### Example 1

### Potentiation of Anti-Tumour Activity of DMXAA in Mice by Thalidomide, PG, PiG and CG

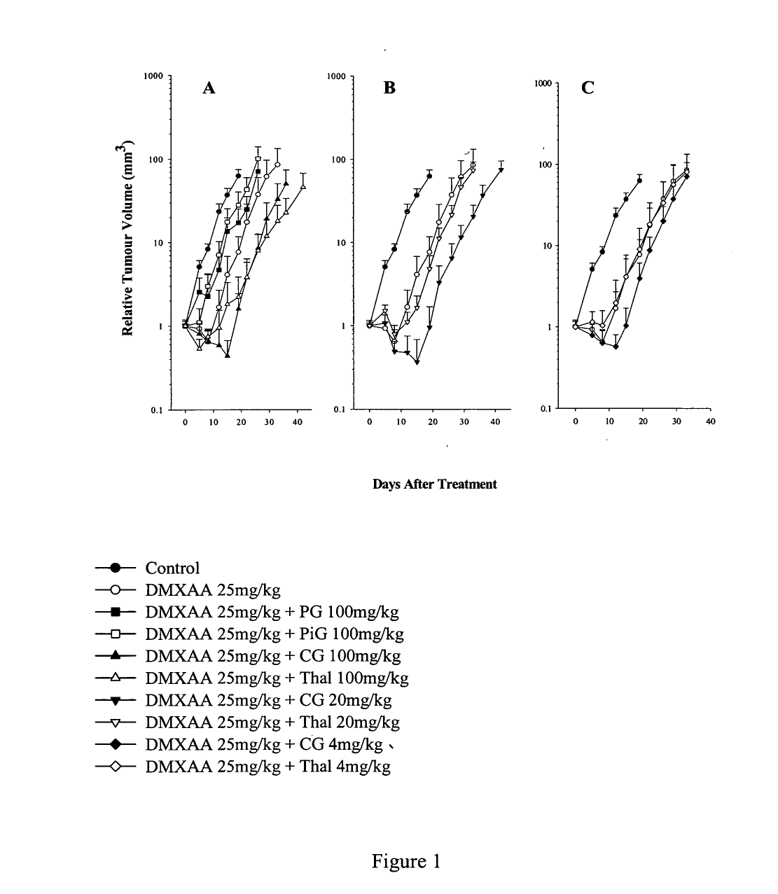

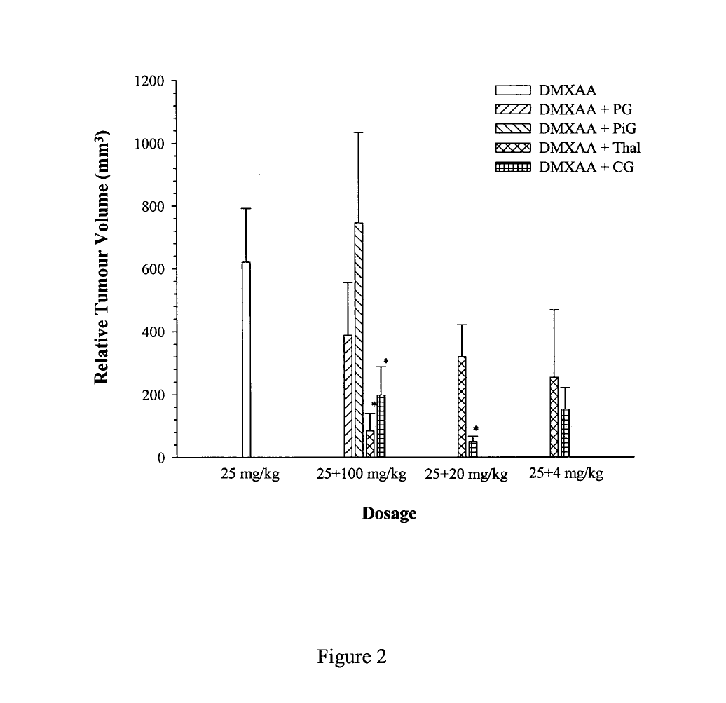

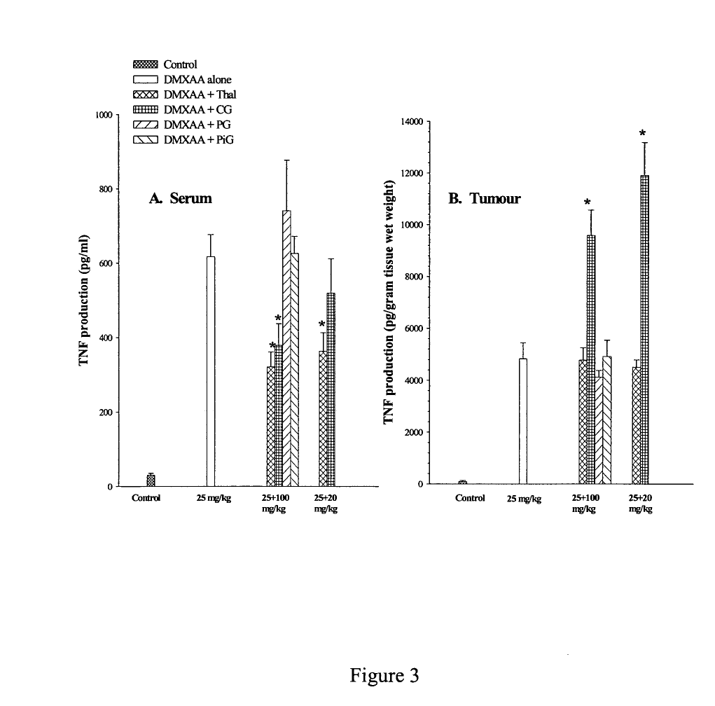

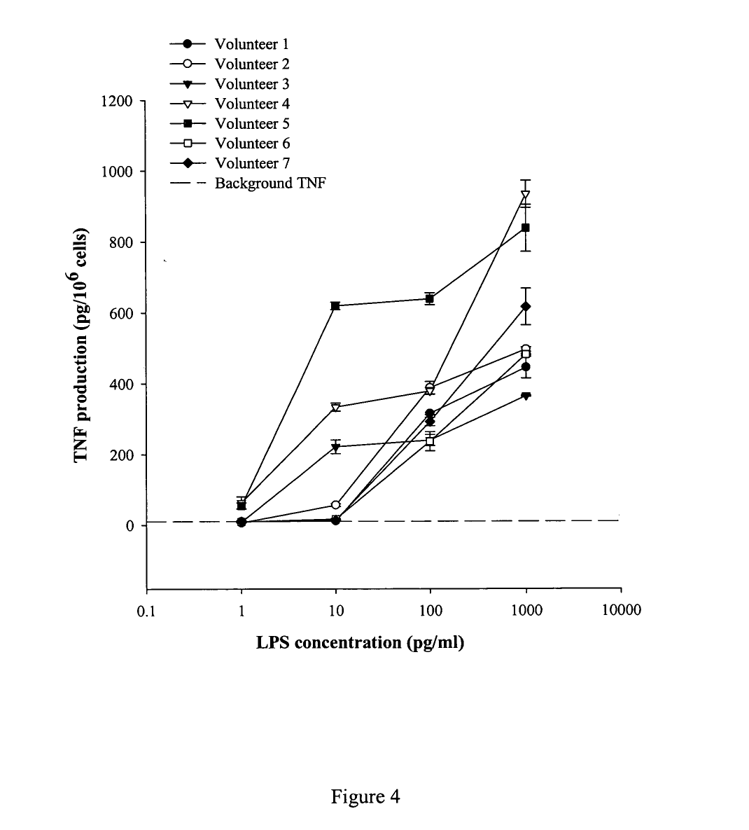

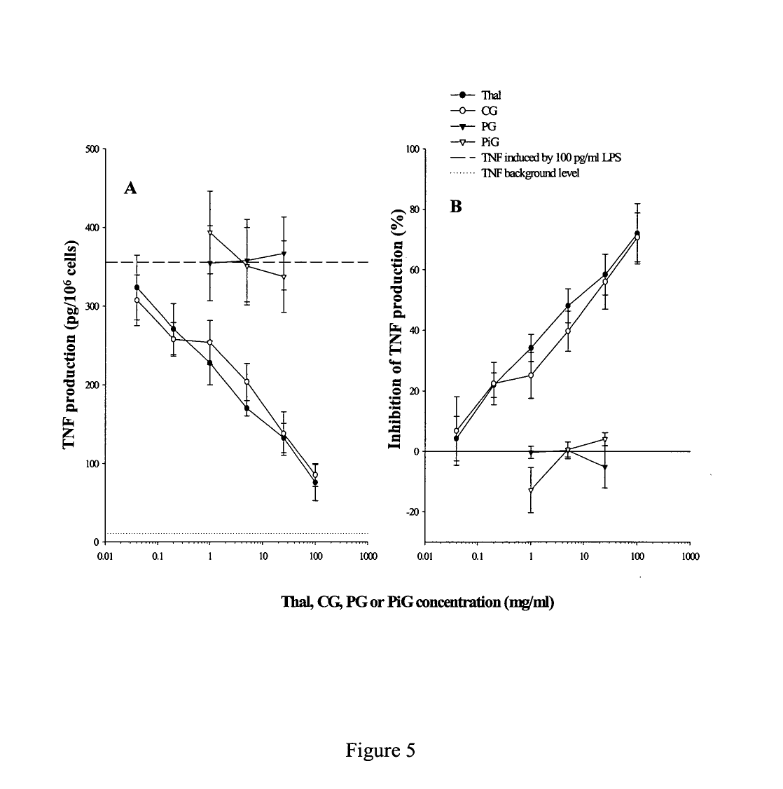

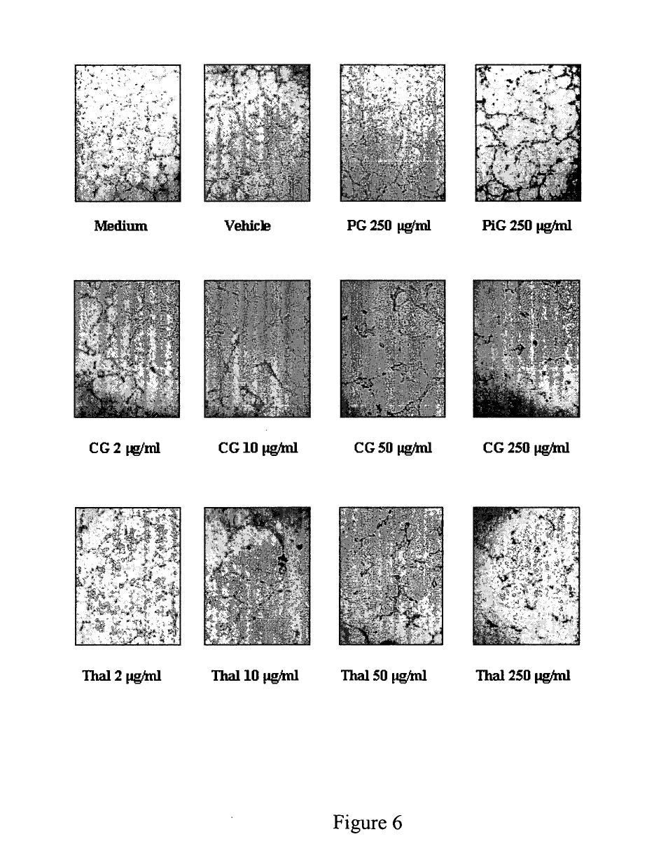

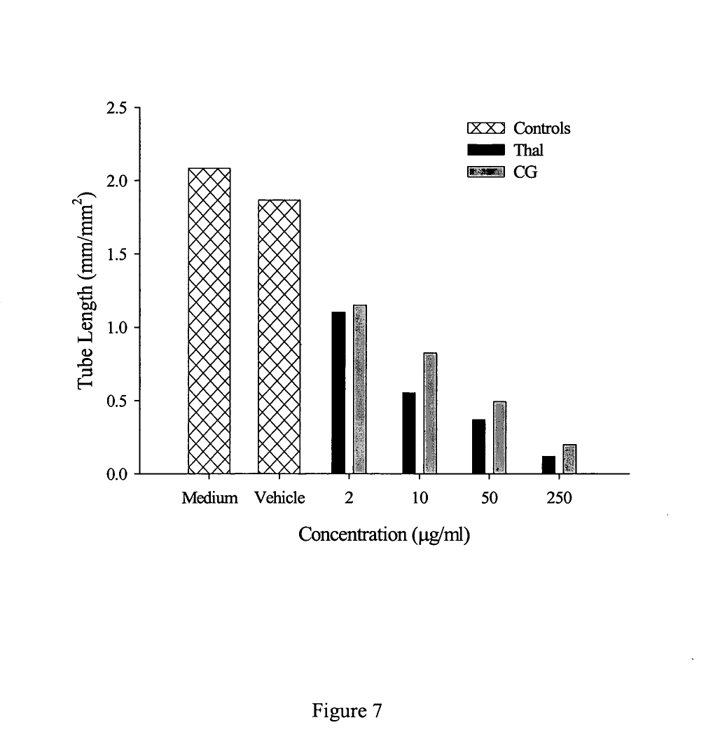

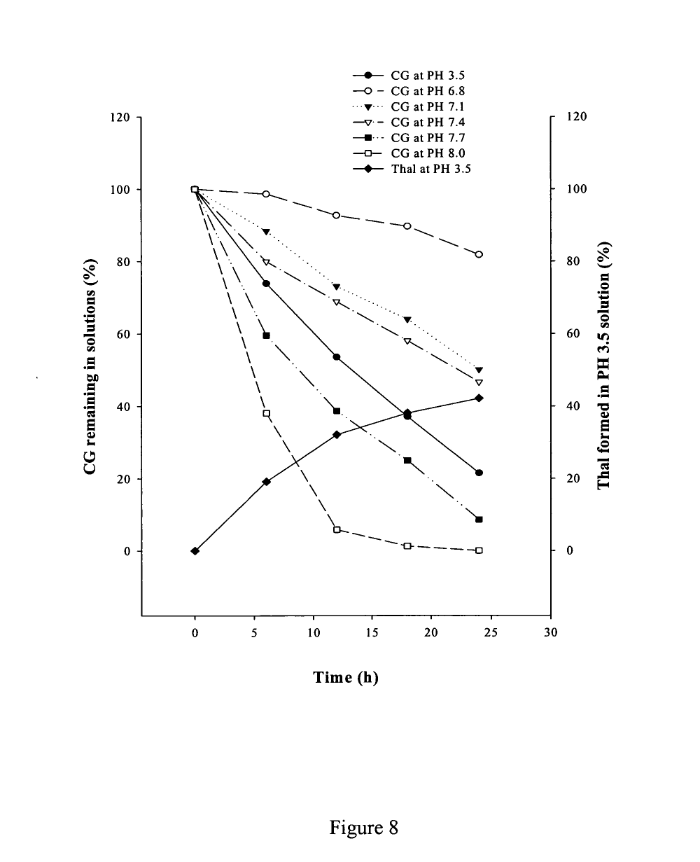

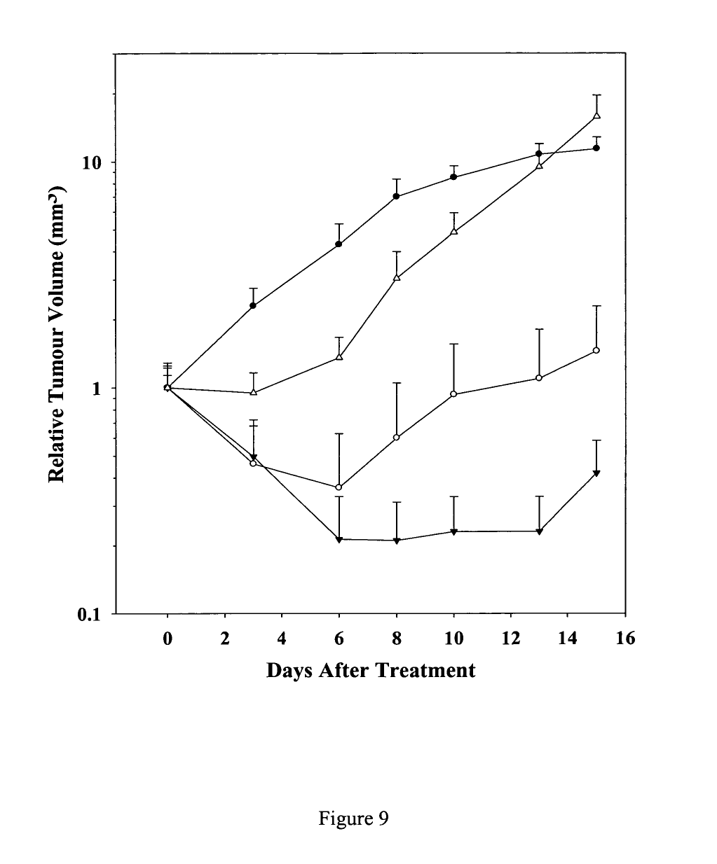

To investigate whether the hydrolysis metabolites of thalidomide had anti-tumour activity and whether or not they could also potentiate the anti-tumour activity of DMXAA, mice with Colon 38 tumours were treated with CG, PG, PiG and thalidomide at 100 mg/kg together with DMXAA (25 mg/kg), and the growth of the tumours was followed. Co-administration of PiG or PG with DMXAA did not inhibit tumour growth above that of DMXAA alone (FIG. 1A). At 21 days after treatment, no significant difference in tumour volumes was observed between the DMXAA-treated group and those treated with DMXAA plus PG or DMXAA plus PiG (FIG. 2). Co-administration of CG however produced greater inhibition of tumour growth than that of DMXAA alone and to a similar extent as thalidomide (FIG. 1A). At 21 days after treatment, mice treated with CG or thalidomide combined with DMXAA had significantly smaller tumour volumes than those treated by DMXAA alone (p<0.05, FIG. 2).

CG was then compared with thalidomide at the lower doses of 20 mg/kg and 4 mg/kg for potentiation of DMXAA activity against Colon 38 tumours. At 20 mg/kg, CG plus DMXAA was more effective than thalidomide plus DMXAA or DMXAA alone (FIG. 1B). At 21 days after treatment, mice treated with a combination of CG and DMXAA had significantly smaller tumour volume than those treated by DMXAA alone (p<0.05). Tumour volumes from mice treated with a combination of thalidomide and DMXAA were similar to those treated by DMXAA alone (FIG. 2). At 4 mg/kg, neither CG nor thalidomide showed potentiation of DMXAA-induced anti-tumour effects FIG. 1C), and there was no significant difference between tumour volumes of mice treated with CG or thalidomide plus DMXAA or DMXAA alone at 21 days after treatment (FIG. 2).

### Example 2

### Effects of Thalidomide, PG, PiG and CG on DMXAA-Induced TNF Production in Mice

Tumour-bearing mice were administered thalidomide, PG, PiG, CG or DMXAA alone, or administered DMXAA (25 mg/kg) plus PG (100 mg/kg), PiG (100 mg/kg), thalidomide (100 mg/kg or 20 mg/kg) or CG (100 mg/kg or 20 mg/kg). Serum and tumour samples of mice were assayed for TNF production 3 h after drug administration. TNF levels in serum and tumours from mice treated with PG, PiG, CG or thalidomide alone were similar to that of untreated mice (student's t-test). Mice treated with DMXAA alone (25 mg/kg) had a 20-fold (FIG. 3A), and 50-fold increase respectively in serum and tumour TNF activity above those of control untreated mice (FIG. 3B). Co-administration of PG and PiG had no effect on DMXAA-induced serum and tumour TNF level (FIG. 3). Co-administered thalidomide reduced DMXAA-induced TNF levels in serum at 100 mg/kg and 20 mg/kg p<0.05, FIG. 3A), but had no significant effect on DMXAA-induced intra-tumoural TNF levels (FIG. 3B). CG significantly increased DMXAA-induced TNF production in tumours at doses of 100 mg/kg and 20 mg/kg (p<0.05, FIG. 3B), and decreased DMXAA-induced TNF levels in serum at 100 mg/kg (FIG. 3A).

### Example 3

### Effects of Thalidomide, PG, PiG and CG on LPS-induced TNF Production by HPBL in Culture

Significant TNF production was obtained when HPBL were cultured with 100 pg/ml of LPS (FIG. 4), and that concentration was chosen for subsequent studies. HPBL Were cultured with LPS plus thalidomide, PG, PiG or CG at various concentrations for 12 h, and the supernatants were assayed for TNF. PG or PiG had no significant effect on LPS-induced TNF production over all the concentrations tested (1, 5, 25 μg/ml, FIG. 5), whereas thalidomide and CG significantly decreased LPS-induced TNF levels in a dose-dependent manner (FIG. 5A) and to a similar extent (FIG. 5B).

### Example 4

### Inhibition of Tube Formation in Matrigel

Tube formation by ECV304 cells on matrigel layers was used as an in vitro model for angiogenesis, and the ability of thalidomide or its hydrolysis metabolites to inhibit tube formation was tested. Near complete inhibition of tube formation was observed with thalidomide and CG at 250 μg/ml (FIGS. 6 & 7), and 38-41% inhibition (FIG. 7) at the lowest concentration tested (2 μg/ml). PG and PiG however, did not show any inhibition on tube formation at all concentrations tested (FIG. 6).

As a control that inhibition of tube formation was not as result of cytotoxicity of the drugs, ECV304 cells were cultured with thalidomide, PG, PiG and CG at the same the concentrations used for tube inhibition (2-250/g/ml), and cell viability after 18 h was measured using the MTT cytotoxicity assay (see above). No toxicity was observed with any of the compounds compared with untreated or DMSO-treated cultures.

### Example 5

### Stability of CG at Different pHs

To examine its stability, CG was incubated at different pHs and at 0, 6, 12, 18 and 24 h after incubation, the concentrations of CG and thalidomide in solution was measured using HPLC assay which resolved CG, phenacetin and thalidomide completely with the retention times of 8.5, 22.8 and 23.6 min respectively. At pH 3.5, 7.7 and 8, 21.5%, 8.6% and 0% of CG was remaining in the solution after 24 h (FIG. 8). CG was more stable at pH 6.8, 7.1 and 7.4. At pH 6.8, 81.75% of CG remained in solution after 24 h of incubation, and at pH 7.1 and 7.4, 50.1% and 46.6% respectively of CG remained in solution. Thalidomide was not detected in CG solutions with pH of 6.8, 7.1, 7.4, 7.7 or 8 after incubation. However, it was detected at pH of 3.5 after 6, 12, 18 and 24 h of incubation (FIG. 8). Since the molar ratio of transformation from CG to thalidomide is 1:1, it was determined that 42% of CG was transformed into thalidomide after 24 h incubation at pH 3.5 (FIG. 8).

The present studies have shown that CG was as active as thalidomide in the in vitro assays of angiogenesis inhibition (FIGS. 6 & 7) and TNF inhibition (FIG. 5) in human cell systems. The inhibition of tube formation was not due to cytotoxicity, as no loss of cell viability was observed at the same drug concentrations, and incubation time. CG performed better than thalidomide in vivo in modulating DMXAA-induced TNF production and anti-tumour responses in mice (FIGS. 1-3). CG provided greater tumour growth inhibition than thalidomide at doses at or above 20 mg/kg when combined with DMXAA (FIGS. 1 & 2). Thus CG possesses anti-angiogenic and cytokine modulatory activities, the two effects that perhaps form the basis of its anti-tumour properties.

The stability studies showed that CG was stable in solution at pHs between 6.8-7.4 (FIG. 8), indicating that the active agent in the biological assays would have been CG and not thalidomide transformed from CG. The stability studies also showed that CG was more stable than thalidomide at physiological conditions, as only 1.3-20% of CG had degraded after 6 h. CG was unstable at low pH however, and 42% had converted to thalidomide at pH 3.5.

### Example 6

### Potentiation of Anti-Tumour Activity of Cyclophosphamide in Mice by Thalidomide, PG, PiG and CG

In example 1, CG was shown to potentiate the activity of the DMXAA. Studies were performed to examine if CG potentiated the anti-tumour response to the anti-neoplastic drug, cyclophosphamide. Studies performed with cyclophosphamide were conducted in accordance with details provided in examples 1 and 2 except that cyclophosphamide (from Sigma Aldrich, St Louis, Mo., dissolved in water) was used in place of DMXAA. The results of these studies are presented in FIG. 9.

Mice with Colon 38 tumours were administered cyclophosphamide alone (220 mg/kg), CG alone (100 mg/kg), or the combination. The growth of tumours in each treatment group was compared with that of untreated tumours. The results (FIG. 9) showed that at day 15 after treatment, tumours treated with the combination were significantly smaller than those treated with each of the monotherapies.

The results of these studies indicate that CG would be an effective anti-tumour agent for clinical use, since it is more soluble, more stable at physiological conditions, and appears to be at least as active as thalidomide in the panel of biological assays described herein. A major advantage of using CG over thalidomide would be that CG appears to be non-teratogenic. CG is able to cross the placenta, but is unable to cause teratogenicity.

Synergistic combinations of, for example, CG and DMXAA or CG and cyclophosphamide provide further novel compositions for effective treatment of tumours in humans and animals. It is clear from studies described herein that other anti-neoplastic agents can be used in combination with CG to achieve the advantageous compositions for the treatment of solid tumours.

Although the present invention has been described with reference to certain preferred embodiments, it will be understood that variations and alternatives which are in keeping with the spirit of the invention are also within its scope.

1. Composition comprising one or more hydrolytic thalidomide metabolites, wherein at least one said metabolite possesses anti-tumour activity, optionally in combination with a pharmaceutically acceptable carrier.

2. Composition comprising one or more hydrolytic thalidomide metabolites, wherein at least one said metabolite possesses anti-angiogenic and/or cytokine modulatory activity, optionally in combination with a pharmaceutically acceptable carrier.

3. Synergistic composition comprising one or more thalidomide metabolites, wherein at least one said metabolite possesses anti-tumour, anti-angiogenic, and/or cytokine modulatory activity, and at least one other anti-neoplastic compound, optionally in combination with a pharmaceutically acceptable carrier.

4. A composition according to any one of claims 1 to 3, wherein the thalidomide metabolite is CG.

5. A composition according to claim 4, wherein the anti-neoplastic compound is selected from the group consisting of DMXAA, cyclophosphamide or variants and analogues thereof.

6. Method of treating a solid tumour in a subject comprising the administration to the subject requiring such treatment a composition of any one of claims 1 to 5.

7. Method of treating a solid tumour in a subject comprising the administration to the subject requiring such treatment a first composition comprising one or more hydrolytic thalidomide metabolites, wherein at least one said metabolite possesses anti-tumour activity, and a second composition comprising at least one other anti-neoplastic compound.

8. A method according to claim 7, wherein the thalidomide metabolite is CG and the anti-neoplastic compound is selected from DMXAA and cyclophosphamide.

9. A method according to claim 7 wherein the first and second compositions are administered sequentially or simultaneously.

10. A method according claim 7, wherein the first and the second compositions are administered by different routes or by the same route.

11. A method according to claim 7, wherein the tumour is selected from the group consisting of colon cancers, breast cancers, liver cancers, lung cancers, brain tumours, ovarian tumours, prostate cancers, testicular cancers and uterine tumours.
